(19) United States
(12) Patent Application Publication
(10) Pub. No.: US 2025/0115969 A1
(43) Pub. Date: Apr. 10, 2025

(54) RESISTANCE AND VIRULENCE DIAGNOSTICS

(71) Applicant: SYNGENTA CROP PROTECTION AG, Basel (CH)

(72) Inventors: Stefano TORRIANI, Stein (CH); Sarah Margaret TARGETT, Bracknell, Berkshire (GB); Paolo GALLI, Basel (CH); Luca CORNETTI, Stein (CH); Lorenzo BORGHI, Stein (CH)

(73) Assignee: SYNGENTA CROP PROTECTION AG, Basel (CH)

(21) Appl. No.: 18/730,361

(22) PCT Filed: Jan. 18, 2023
(86) PCT No.: PCT/EP2023/051107

§ 371 Date: Jul. 19, 2024

(30) Foreign Application Priority Data
Jan. 19, 2022 (EP) 22152290.7

(51) Int. Cl. C12Q 1/6895

(52) U.S. Cl. CPC C12Q 1/6895; C12Q 2600/156

(57) ABSTRACT

A method for determining a resistance and/or virulence profile for a plant pathogen in a location. The method comprising receiving a sample of a plant pathogen, subjecting the sample to DNA sequencing, determining the presence of at least one genetic variance of the plant pathogen and quantifying said at least one genetic variance based on the DNA sequencing, and generating a resistance profile and/or virulence profile of the plant pathogen.

### TECHNICAL FIELD

The present disclosure relates to a method for determining resistance and/or virulence profile for a plant pathogen in a location. This can be useful for providing real-time or close to real-time tailored recommendations of control disease measures.

### BACKGROUND

Plant pathogens are organisms that cause disease in a plant. This may result in reduced plant growth, plant assimilation or even impairment of the vital functions of the plant resulting in a reduction or loss of plant productivity. Plant pathogens include viruses, bacteria, nematodes, insects, and fungi, and they are capable of reproducing within or on its host spreading from one plant to another. Symptoms of a plant disease include change in color, shape, or function, for example the fungi Zymoseptoria tritici causes pale brown to greenish-grey oval lesions in the leaves of wheat. In case of moderate to high disease pressure, it can cause significant lost in plant productivity.

All species of plants, cultivated and wild, can be susceptible to disease. For farmers, plant pathogens may reduce yield and quality of agricultural production causing substantial economic loss. As such, there has been extensive research and development in managing or controlling plant disease including crop rotation, appropriate planting date and plant density, control of field moisture, breeding plants with greater resistance to pathogens, and application of pesticides or biopesticide.

Pesticides are chemical or biological agents used for protecting plants against pathogens. Fungicide is a type of pesticide which can be a chemical compound, biological organism or derivates of biological organisms that are sprayed onto crop, soil or seed, inhibiting the development or killing fungi and/or their spores. There are various types of fungicides which affect pathogens in different ways. The chemical structure of the active ingredient (AI) fungicide and the target inhibited usually defines its mode of action, in other words, how it interacts with a pathogen. For example, one type of fungicide may bind to enzymes involved in nucleic acid metabolism of the fungi such that the reproduction and the pathogen's ability to control key aspects of its biology are disrupted. Another type of fungicide inhibits the biochemistry pathways involved in converting nutrients into the energy needed for a pathogen to grow. An example of such a fungicide class is succinate dehydrogenase inhibitors (SDHIs) which inhibit the cellular respiration and energy production of the fungi by targeting the enzyme succinate dehydrogenase.

Although fungicides can be very effective in controlling a plant disease, pathogens respond to fungicides by evolving resistance meaning that the fungicide become less effective. Resistance of a pathogen to a control agent, such as a fungicide, can occur through mutation of a single or several genes of the pathogen. The mutation(s) may result in various mechanisms in which the pathogen becomes resistant or less sensitive to a fungicide. For example, mutations may result in that the pathogen has an altered target site such that the fungicide can no longer, or less effectively, bind to it. Beside target modification, other resistance mechanisms can occur. A pathogen developing efficient efflux mechanism pumping the fungicide out of its cells, or the pathogen overexpressing the target protein can develop resistance or decreased sensitivity. The resistance mechanisms may have a sudden or gradual effect on the ability of the pathogen to overcome the effect of the fungicide. If there is a sudden mutation, then the pathogen may develop a complete resistance to a particular fungicide such that the fungicide is no longer effective. If there is a gradual effect, then there is an accumulation of many mutations in different genes, each contributing in a gradual manner to the resistance of the pathogen, such that an increasing shift in sensitivity of a pathogen to a fungicide can be observed over several years. The sensitivity of a pathogen to a particular fungicide may continue to reduce if the fungicide is continuously applied to control a particular pathogen which may lead to resistance.

For example, fungicides containing quinone outside inhibitors (QoI), demethylation inhibitors (DMI) or succinate dehydrogenase inhibitors (SDHI) have been widely used in the last decades for Zymoseptoria tritici control in Europe and other geographic regions of the globe.

QoIs have lost their ability to control the pathogen due to the development of a single mutation in the cytb gene, resulting in an amino acid change, identified as G143A, which confers a complete resistance to the pathogen. Nowadays, this mutation is widespread in most cereal growing regions in Europe and the contribution of QoI for the control of Zymoseptoria tritici is minimal.

Over several years the activity of another fungicide class, the DMI fungicides, have eroded with time due to a sensitivity shift of Zymoseptoria tritici. The mechanism behind this erosion is mainly attributed to an accumulation of different mutations in the cyp51 gene of the pathogen. It is to note that different mutations or combinations of mutations can confer different degree of resistance to different fungicides within the DMI group. This effect is called incomplete cross resistance as reported by the Fungicide Resistance Action Committee, FRAC. Therefore, the efficacy of a specific DMI fungicide can vary depending on the resistance profile of the pathogen in a specific location in response to the local distribution of individuals harboring different type of resistance features.

Resistance to SDHIs has been reported in more recent years. For example, in Zymoseptoria tritici different mutations in the subunits B, C and D of the sdh genes (which is part of the ubiquinone and SDHI binding site of the complex II) have been found reducing the pathogen sensitivity to SDHI fungicides. Monitoring data published on the FRAC website indicates that mutations like B-N225T, C-T79N, C-W80S, C-N86S and C-H152R can confer different degree of resistance to Zymoseptoria tritici. Incomplete cross resistance to SDHI has been observed. Moreover, it is known that some members of a fungicide class can maintain a significant performance despite the presence of the resistant individuals in a field by showing a higher efficiency to bind the target resulting in a higher potency.

New fungicides with different mode of action have been introduced to the market in recent years like the Quinone Inside Inhibitor (QiI) and the Quinone outside Inhibitor (QoI) with the ability to control pathogens bearing the G143A mutation (classified as 11A group by FRAC). Single point mutations have been demonstrated to affect the performance of these new fungicides and pathogens might evolve to gain the ability to overcome the fungicidal activity of these new fungicides. For example, laboratory studies indicated that a single mutation in the cytochrome b (G37V) could confer the ability to Zymoseptoria tritici to overcome the fungicidal effect of quinone inside inhibitors (QiI), like fenpicoxamid and florylpicoxamid.

### SUMMARY

As discussed in the background section, pathogens can develop resistance towards a pesticide such that the pesticide becomes less effective, or even ineffective, in managing the infection, reproduction and spread of the pathogen. Also, virulence of a pathogen can change as a result of mutation and also differ depending on the plant variety. As such, disease control and resistance management of pesticides need to be carefully implemented to avoid fast selection of resistance leading to ineffectiveness and diminished options available to a farmer, agriculturalist, agronomist or the like for controlling the pathogen.

The present disclosure at least partially aims to address this challenge.

According to an aspect of the present disclosure, a method for determining a resistance and/or virulence profile for a plant pathogen in a location is provided. The method comprises receiving a sample comprising a plant pathogen, subjecting the sample to DNA sequencing, determining the presence of at least one genetic variance of the plant pathogen and quantifying said at least one genetic variance based on the DNA sequencing; and generating a resistance profile and/or virulence profile of the plant pathogen. Thus, the method can provide a diagnostic of a plant or field of plants in terms resistance development and/or virulence of a plant pathogen. This can be particularly useful in order to understand the development of resistance and virulence of pathogens at a location. It can also be useful for determining a more tailored disease control measure and/or resistance management program for a current season and also future seasons. For example, the most effective type of fungicide for controlling the pathogen and/or the best crop variety can be selected within a season or for a future season such that fungicides with high presence of resistance can be avoided.

The DNA sequencing of the method may provide a plurality of genetic variances and associated quantity of the plant pathogen in a single read. This means that some or all variances as well as combinations of all variances can be monitored at the same time. Furthermore, the quantification of each identified variance and genotype provides a more accurate understanding of the plant pathogen or plant pathogen population such that a more tailored treatment program for disease control and resistance management can be provided.

Generating a resistance profile may comprise associating the at least one genetic variance and corresponding quantity with a level of resistance to a pesticide and/or a group of pesticides. Additionally, or alternatively, generating a virulence profile may comprise associating the at least one genetic variance and corresponding quantity with a level of virulence. This provides a much better overarching understanding of the resistance and/or virulence of a pathogen or plant pathogen population present in a plant or a location such as a field of plants.

The method may further comprise determining at least one disease control measure for controlling the development, reproduction and/or viability of the plant pathogen based on the resistance profile and/or virulence profile. This is particularly useful for a farmer, agriculturalist, agronomist or the like trying to control a disease present in a field as the disease control measure can be tailored to the specific variant(s) of the pathogen present in the field whilst managing resistance. For example, the determined disease control measure may be at least one pesticide, wherein the pesticide may be at least one fungicide.

The method may further comprise receiving a subsequent sample comprising the plant pathogen that has been subjected to at least one disease control measure, subjecting the sample to DNA sequencing, determining the presence of at least one genetic variance of the plant pathogen and quantifying said at least one genetic variance based on the DNA sequencing, generating a resistance profile and/or virulence profile of the plant pathogen, and comparing the resistance profile and/or virulence profile of the subsequent sample with that of the preceding sample. This can give insight into the success of the disease control measures applied. For example, it can indicate that the disease control measures applied were suboptimal or successfully controlled the plant pathogen whilst controlling resistance development. Additionally, information about the disease control measures such as date of application, plant protection product applied, rate applied and other information can be shared and thereby enable alerts about a risk decision and propose alternatives for the following seasons.

The sample may be collected at a tillering stage of a plant hosting the plant pathogen and the subsequent sample may be collected at a fruit development stage of said plant. This enables an appropriate disease control measure to be determined and applied early in the season and the effectiveness of the applied disease control measure to be assessed.

The sample and the subsequent sample may be obtained from a plurality of plants, spore traps and/or soil. This enables various types of pathogens to be analysed and recommendations to be made.

The sample and/or subsequent sample may be obtained from the location, and the method may further comprise determining at least one disease control measure for applying to a neighboring location based on the resistance and/or virulence profile. This means that in some cases it can be assumed that the variant(s) of the plant pathogen present in the location are also present in neighboring locations such that the disease control measure determined for the location can also be effective in neighboring locations without having to determine a resistance and/or virulence profile based on a sample from any of the neighboring locations.

Subjecting the sample to DNA sequencing may comprise using a sequencer capable of sequencing at least 200 base pairs, preferably at least 500 base pairs, in a single read and/or configured to process at least 100 reads in a single experiment or run. Such a sequencer may use nanopore sequencing technology and/or single-molecule real-time sequencing. This provides the advantage that the samples can be analysed rapidly and give a representation of the disease progression in real-time, close to real-time, instantaneously or near instantaneously. As such, recommendations including disease control measures and resistance management can be implemented within season. This provides more effective or optimal recommendations compared to recommendations based on historic data.

The method may further comprise determining, based on the DNA sequencing, the presence of sdhC_079N, sdhC_086S and/or sdhC_H152R genetic variants in plant pathogen Zymoseptoria tritici hosted by a wheat plant, wherein the method further comprises recommending a resistant wheat variety and/or a spray program comprising high intrinsic active SDHI or at least one fungicide with different mode of action including florylpicoaxamid, fenpicoxamid, metyltetraprole, mefentrifluconazole and/or prothioconazole as a disease control measure.

The method may further comprise determining, based on the DNA sequencing, the presence of cyp51_379G, cyp51_381V, cyp51_460, cyp51_524T genetic variants in plant pathogen Zymoseptoria tritici hosted by a wheat plant, wherein the method further comprises recommending a resistant wheat variety and/or a spray program of high intrinsic active DMI or at least one fungicide with different mode of action including florylpicoxamid, fenpicoxamid, metyltetraprole, isoflucypram, fluxapyroxad, benzovindiflupyr or pydiflumetofen as a disease control measure.

In the method, the step receiving a sample comprising a plant pathogen may comprise receiving from a crop field a crop sample comprising a plant pathogen.

In one example, a user such as a farmer, agriculturalist, agronomist or the like, collects the sample and then sends it on to an organisation, company or person carrying out any of the methods described herein and then the user receives a resistance and/or virulence profile, and/or at least one disease control measure for controlling the development, reproduction and/or viability of the plant pathogen based on said resistance profile and/or virulence profile.

### BRIEF DESCRIPTION OF THE DRAWINGS

For a better understanding of the present invention, and to show more clearly how it may be carried into effect, reference will now be made, by way of example, to the following drawings in which:

FIG. 1 is a flow diagram of an example method of the present disclosure;

FIG. 2 is an illustration of an example of genetic variants of a plant pathogen population and corresponding frequencies present in a sample (SDHI case);

FIG. 3 is an illustration of an example of genetic variants of a plant pathogen population and corresponding frequencies present in a sample (DMI case);

FIG. 4 is a flow diagram of an example method of the present disclosure;

FIG. 5 is another flow diagram of an example method of the present disclosure; and

FIG. 6 is yet another flow diagram of an example method of the present disclosure.

### DETAILED DESCRIPTION

In the following description, for purposes of explanation, numerous specific details of certain examples are set forth. Reference in the specification to “an example” or similar language means that a particular feature, structure, or characteristic described in connection with the example is included in at least that one example, but not necessarily in other examples.

As discussed in the background section, pathogens can develop resistance towards a fungicide such that the fungicide becomes less effective, or even ineffective, in managing the infection, reproduction and spread of the pathogen. As such, disease control and resistance management of fungicides needs to be carefully implemented to protect the plant and to avoid fast selection of resistance leading to ineffectiveness and diminishing options available to the farmer, agriculturalist, agronomist or the like for controlling the disease.

Resistance management of fungi in crops currently involves applying a combination of fungicides with a different mode of action at specific time points throughout the crop growing season based on product availability for the control of a specific pathogen. Approximate knowledge of the presence of resistance of a pathogen in a region or country and general recommendations are today provided, however, this only offers general information on the presence of resistance and recommendations on how to deal with it. Examples of the present disclosure provide methods of how resistance management can be improved and tailored to a specific situation. This may be achieved by determining a resistance and/or virulence profile for a plant pathogen through qualitative and quantitative pesticide (e.g., fungicide) sensitivity of the plant pathogen to a specific pesticide and/or qualitative and quantitative virulence of a plant pathogen to a crop in a specific location. This can be determined rapidly, for example in real-time, close to real-time, instantaneously, near instantaneously or within 48 hours as is explained in more detail below. By knowing the genetic variances of a plant pathogen population and their respective quantity or frequency at a specific location and at a given time, a more tailored treatment program or disease control measure can be provided within season that also considers resistance development of the plant pathogen. It may also be combined with a recommendation of an optimal or particular resistant crop. This will allow for a more sustainable use of the tools available for pathogen management and avoid non-effective solutions for that particular location. In addition, it can help to improve the agronomic practice in order to deliver the best control of the pathogen and a sounder resistance management strategy.

Terminologies used herein will now be described.

The terms crop and plant are interchangeably used in the present disclosure and they refer to any type of plant that is cultivated or growing naturally under different conditions like open field or greenhouses. For example, a crop or a plant may be a cereal, fruit, vegetable, tree, flower, grass and/or bush.

As used herein, a field, crop field or a field of crop(s) or plant(s) refers to an area or location where crop grows naturally or is cultivated under different conditions like protected (e.g. greenhouse) or open field conditions.

Disease control measure or treatment program is to be understood as management of a pathogen, for example, by application of a plant protection product such as a pesticide, timing or intervals of application of a plant protection product or selection of an optimal or particular resistant crop in order to control the development, reproduction and/or viability of the pathogen whilst minimizing resistance development.

A “sample” as used herein can be obtained by collecting at least some plant material or at least one environmental sample (aerial or soil sample) surrounding at least one crop. Therefore, a sample may be a field sample such as a leaf, several leaves and/or other part of the crop that is collected from a crop or a field or alternative host plants (e.g. crop wilt type forms or volunteer crops). Additionally, or alternatively, a sample may be an environmental sample comprising soil and/or aerial material collected from where a single crop grows, or collected from several points across a field or location. In one example, a “sample” is to be understood as a sample of a plurality of plants, a spore trap or soil. The sample comprises at least one plant pathogen and should be understood as a representation of a local population of the at least one plant pathogen.

A genetic variance, gene variance or a variance of a plant pathogen is to be understood as a mutation in the DNA sequence different from the wild-type sequence. For example, in a genetic variance at least one nucleotide in a DNA sequence has been permanently changed, deleted or inserted.

Genotype refers to the unique combination of multiple genetic variances, gene variances or variances of a plant pathogen. If multiple independent genetic variations can be brought together into different unique combinations, then each of these unique combinations might express a different sensitivity or virulence profile.

As used herein plant pathogen, also known as crop pathogen or just pathogen, refers to a living organism negatively affecting the structure, development and/or vital functions of a plant. For example, a plant pathogen may be a virus, viroids, fungi, nematodes, bacteria, phytoplasm, protozoa, algae, insect and/or parasitic plants. The term plant pathogen should be understood to mean an individual organism or several organisms of the plant pathogen.

A plant pathogen population as used herein refers to a representation of individual plant pathogens present in a location.

Sensitivity, or fungicide sensitivity, or pesticide sensitivity, refers to the susceptibility of a plant pathogen to a pesticide such as a fungicide. Variations of sensitivity to fungicides can result from a range of different mechanisms as described in the background section, for example mutations in the genome.

Resistance, or fungicide resistance, or pesticide resistance, refers to the ability of a plant pathogen to survive the exposure to pesticides designed to control it. As for sensitivity, several mechanisms, such as those described in the background section, can shape the resistance or adaptation level. In contrast to sensitivity, resistance of a plant pathogen means that the plant pathogen becomes less sensitive to such an extent that a pesticide such as a fungicide is no longer effective in controlling the development, reproduction and viability of the plant pathogen.

Resistance development refers to a plant pathogen which develops a lower sensitivity to a fungicide or pesticide. Lower sensitivity can result in complete resistance or gradual resistance to a fungicide or pesticide. A single mechanism or a combination of mechanisms can shape resistance development. The resistance development depends on how fast these mechanisms are selected in a population of time and space.

A resistance management program used herein should be understood as measures that can be adopted to minimize development of resistance of a pathogen to a plant protection product, such as a pesticide.

As used herein, resistance profile refers to the ability of a plant pathogen or a population of a plant pathogen to overcome and survive the exposure to a pesticide, fungicide, a pesticide class or fungicide class as defined by FRAC, or other chemical or biological agent used for controlling development, viability and reproduction of the plant pathogen.

As used herein, virulence profile refers to the ability of a plant pathogen or a population of a plant pathogen to infect and cause damage to a host, wherein the host may have a type of built in resistance to the pathogen (resistant traits).

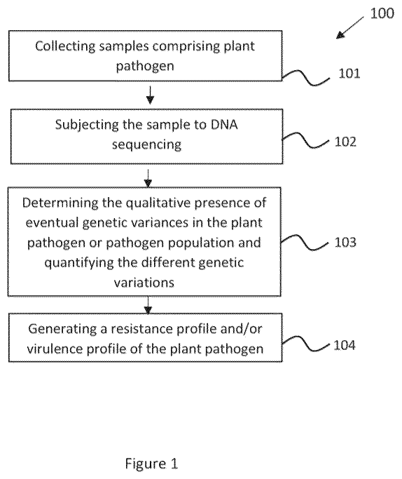

Referring to the figures, FIG. 1 illustrates an example method 100 for determining a resistance profile and/or virulence profile in a plant pathogen population present in a location according to the present disclosure. The method comprises collecting samples from the location 101 wherein the samples could be a field sample or an environmental sample, for example an aerial sampling by spore traps, plant material or soil material as discussed above. Samples are then sent to a laboratory for DNA sequencing 102 in order to determine the genetic sequence of bases (A, C, G and T) known to possibly confer reduced sensitivity to fungicides or pesticides or confer a higher virulence to a resistant plant or cultivar. The method further comprises determining the qualitative presence of genetic variances in the plant pathogen or pathogen population and quantifying the different genetic variations 103. Thereafter, the method further comprises generating a resistance profile and/or virulence profile of the plant pathogen or pathogen population 104 by combining the results with information or knowledge obtained from dedicated studies assessing the impact of a given mutation on the performance of a specific fungicide or pesticide, or by evaluating the possible impact on crop productivity in case of increased virulence to the crop.

Method 100 enables the quantitative and qualitative description of the resistance profile and/or the virulence profile of a population of a pathogen present in a location to be determined rapidly such as in real-time, close to real-time, instantaneously, or near instantaneously, within 48 hours or a few days. This provides opportunities to have a clear and more detailed understanding of the possible resistance profiles present in a location and to help determining a more tailored disease control measure and/or resistance management program for the current season and/or future seasons. As such, the most effective type of pesticide(s) for controlling the pathogen and or the best crop variety can be selected such that pesticides with high presence of resistance can be avoided.

Referring now again to the operation of collecting a sample of a plant pathogen 101, this operation may comprise receiving a sample from a crop field, or a farmer, agriculturalist, agronomist or the like who has collected the sample from a crop field. The sample may alternatively be received from another source, e.g., a wild population of spontaneous crops or flowers, where the profile of resistance of a plant pathogen population is to be determined. The sample may be specimens collected from various points or plants across a field or wild population of spontaneous crops or flowers. Alternatively, or additionally, a sample may be environmental (e.g., soil and/or aerial samples).

The operation of subjecting the sample to DNA sequencing 102 for determining the nucleic acid sequence in the sample will now be described in more detail. This operation may comprise using a sequencer capable of sequencing at least 200, 300, 400 or 500 base pairs in a single read. For example, Oxford Nanopore sequencing Technologies such as MinION, GridION or PromethION or PacBio Sequel Systems implementing single-molecule real-time sequencing as provided by PACBIO may offer the required capability. These technologies may be referred to as third generation sequencer and they provide a high throughput combined with larger sequenced genetic regions from a few hundred base pairs up to 10,000 base pairs. The technology provided by Oxford Nanopore Technologies comprises flow cells which contain an array of tiny holes referred to as nanopores that are embedded in an electro-resistant membrane. Each nanopore corresponds to its own electrode connected to a channel and sensor chip, which measures the electric current that flows through the nanopore. When a molecule passes through a nanopore, the current is disrupted to produce a characteristic ‘squiggle’ or a current intensity value. The squiggle is then decoded using base calling algorithms to determine the DNA. Base calling is a computational process of translating the squiggle into DNA sequences. Specific bioinformatics pipelines may be combined to enable quantification of genetic variants in pathogen populations.

Before subjecting a sample to DNA sequence, DNA may be extracted from the received sample. This DNA sample may include a diversity of individuals from a plant pathogenic species representative of genetic variation of the disease. Genes of interest, for example a fungicide target, virulence or population genetics loci, may then be amplified from the total DNA sample by single step or multiplex polymerase chain reaction (PCR) process. In one example, specific panels of multiplex PCR are designed to co-amplify multiple targets in a single PCR reaction. This optimized step requires the identification of non-self-cross-hybridizing primers in conserved regions to cope with natural variability between individuals composing a population. Through this optimization genetic loci are amplified from the largest fraction of strains in a natural population. Primers may also be checked to ensure that other plant pathogens that could occur in the same host plant are not unspecific amplified. Thereafter, DNA barcoding may be performed to identify a specific sample if multiple samples are bulked before sequencing the plant pathogen. Thereafter, sequencing preparation may be performed followed by the operation of DNA sequencing 102 using a third-generation sequencer as described above.

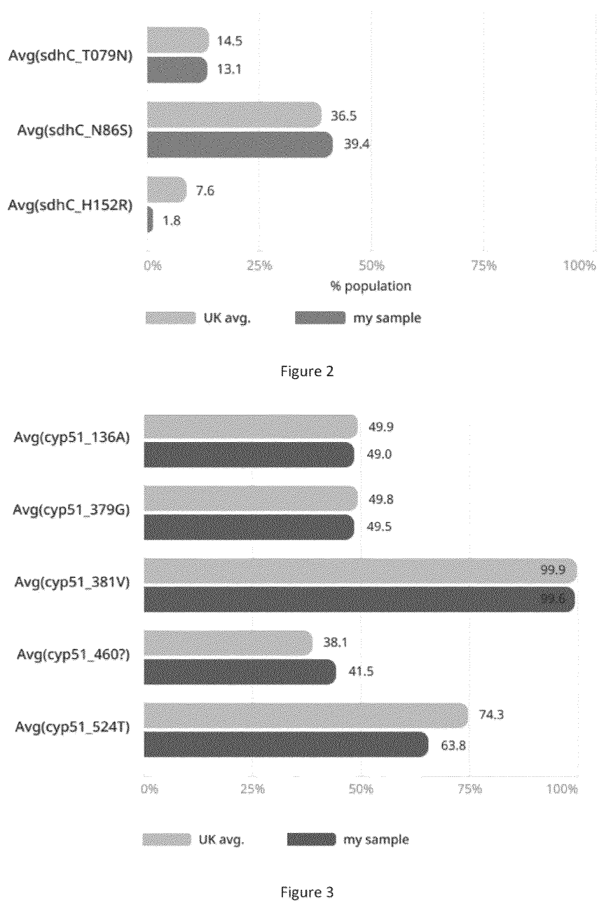

It should be understood that the present disclosure is not limited to the specific third generation sequencers as described herein but that any sequencing technology can be applied that generates data within a timeframe giving a current insight into a disease of a plant. Referring now again to the operation determining the presence of genetic polymorphisms based on the DNA sequencing 103. This operation may comprise comparing the DNA sequence of the sample with a reference DNA sequence of a plant pathogen and identifying genetic polymorphisms. For example, in Zymoseptoria tritici so far at least 25 genetic variants have been monitored for SDHI. Among this complexity the most frequent amino acidic polymorphisms identified to influence sensitivity to SDHI are sdhC-T79N, sdhC-N86S or sdhC-H152R. Quantification of all known variants can be expressed as frequency and this may be determined as a percentage of the whole population of the plant pathogen present in the sample. An example of this is illustrated in FIG. 2, which shows a report or profile of the three major variants in a plant pathogen population associated with SDHI decreased sensitivities. In the specific sample of FIG. 2, sdhC-T79N frequency is 13.1% meaning that 13.1% of the plant pathogen population in the sample have the specific genetic variance defined as sdhC-T79N. Similarly, sdhC-N86S and sdhC-H152R have a frequency of 39.4% and 1.8%, respectively. The frequency of the genetic variances can also be expressed in alternative ways, for example, heatmaps, boxplots, or pies associated with or without maps. The performance of a pesticide is expected to be more affected in situations of high frequency of specific resistant individuals within a pathogen population. Similarly, populations with high frequency of individuals with high virulence determinates are expected to infect resistant crops more efficiently. In the example shown in FIG. 2, the frequency of single variants is presented. It should be understood that the frequency of possible combinations of single genetic variants, a so-called genotype, can additionally or alternatively be provided.

FIG. 3 illustrates another example of frequencies of genetic variances of a sample containing Zymoseptoria tritici and the sensitivity to the fungicide class of DMI. Several genetic variances have been described to influence sensitivity to DMI. For example, in FIG. 3, 49% of the plant pathogens in the sample has variant cyp51_136A, 49.5% of the plant pathogens in the sample has variant cyp51_379G, 99.6% of the plant pathogens in the sample has variant cyp51_381V, 41.5% of the plant pathogens has variant cyp51_460, and 63.8% of the plant pathogens in the sample has variant cyp51-524T. As is shown in the examples in FIG. 2 and FIG. 3, a plant pathogen can comprise more than one genetic variance. In addition, combinations of different variants in a single individual can also be detected and quantified following the process described above (example not shown).

Referring now again to the operation of generating a resistance profile and/or virulence profile of the plant pathogen 104 as shown in FIG. 1. This operation may comprise interpreting the data determined by DNA sequencing by applying knowledge of different genetic variances and their resistance ability to a given pesticide or infection ability (virulence) on a resistant crop. More specifically, generating a resistance profile may comprise associating the genetic variance and corresponding frequency in the plant pathogen population of the sample as determined in the preceding operation 103 with a level of resistance to a pesticide and/or a group of pesticides. For example, variant sdhC-T79N and variant sdhCN86S contribute less to the level of resistance to SDHI. In other words, these variants are associated to low resistance factors. On the other hand, variant sdhC-H152R shows higher resistance factors to SDHI and therefore contributes more to the overall resistance profile to SDHI of a pathogen population compared to the previous mentioned variants. As such, depending on the variants present in the sample and their quantity or frequency, an overall resistance level to SDHI fungicides can be determined. The resistance level can be considered to be an index, which condenses complex information of frequencies and resistance factors of each variants to a single value comparable between different samples. The resistance level of a plant pathogen population of a sample requires an understanding of the effect of each variant to the sensitivity of a pesticide, or more specifically a fungicide.

As described above, operation 104 may alternatively or additionally comprise generating a virulence profile based on the determined at least one genetic variance and its corresponding frequency. The virulence profile indicates the plant pathogen's or a population of the plant pathogen's ability to invade a host and cause damage to different crop varieties. For example, several wheat cultivars exist, each with a particular susceptibility to Zymoseptoria tritici. To date, 21 wheat genes (Stb genes) conferring resistance to Zymoseptoria tritici have been identified. A well-documented example is wheat gene Stb6 conferring resistance to Zymoseptoria tritici which is in direct interaction with AvrStb6 in Zymoseptoria tritici, wherein AvrStb6 modulate virulence. The virulence profile may indicate a virulence level of the population of plant pathogens in a sample. The virulence profile may be an index in that the generated data indicating the level of virulence is normalized so that it can be compared with other samples.

It should be understood from the above description that a resistance profile and a virulence profile may be generated alone or in combination for a single sample.

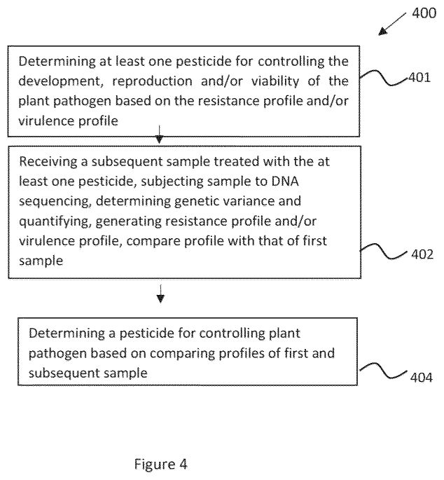

FIG. 4 illustrates a method 400 comprising further optional operations of method 100. These operations follow on from operation 104 of method 100.

Method 400 comprises determining at least one pesticide or other disease control measure for controlling the development, reproduction and/or viability of the crop pathogen based on the resistance profile 401. For example, a resistance profile and/or virulence profile may be determined early in the season before any use of pesticides to control Zymoseptoria tritici. The resistance profile to specific fungicide classes (e.g. SDHI or DMI) of the pathogen population can be generated or measured as described above in example method 100 by monitoring the variants present in the sample and quantify their frequency. Similarly, a virulence profile can be generated as described above. Based on the resistance profile and/or virulence profile a pesticide or other disease control measure can be determined or recommended. The method 400 may further comprise an operation 402 of collecting or receiving a subsequent or additional sample from the same location, crop field, crop or plant as the initial sample, wherein the subsequent sample has been treated with the pesticide or other disease control measure determined in operation 401. The subsequent sample is subjected to the operations 102 and 103 described above with reference to FIG. 1, and thereafter a resistance profile and/or virulence profile is generated similar to operation 104. Thereafter, the generated resistance profile and/or virulence profile is compared with that of the initial sample. For example, if a resistance profile was generated for the initial sample then it is compared with a resistance profile generated for the subsequent sample, similarly if a virulence profile was generated for the initial sample then it is compared with a virulence profile of the subsequent sample. From the comparison of the two resistance profiles, it is possible to determine if the population resistance to a fungicide class improved or deteriorated as a consequence of the agronomic decisions taken to control the disease. For example, the ratio between a sensitivity index generated before and after a pesticide treatment provides information about the sustainability of the disease control measure applied and can be used as a tool to identify and inform about suboptimal disease control measures and resistance management. The sharing of information about the disease control measures such as date of application, plant protection product applied, rate applied and other information will enable alerts about a risk decision and propose alternatives for the following seasons.

In one example, based on the comparison, the method 400 may further comprise determining at least one pesticide or other disease control measure for controlling the plant pathogen. This pesticide or other disease control measure may be the same or different to the one(s) applied before the subsequent sample was collected and received.

The first or initial sample described herein may be collected at the beginning of the season and the subsequent or additional sample may be collected during or at the end of the season (middle or end). The beginning and the middle/end of the season may be a period of a day, several days, a week or several weeks, a month or several months, a year or several years depending on the crop, pathogen or sampling frequency needed for the particular situation. For example, for wheat in UK the first sample collected at the beginning of the season could be in March, before any pesticide treatment, and the subsequent sample collected at the end of the season could be in July, a few weeks after completion of a spray program. In the event that a more frequent analysis of a resistance profile is required, other samplings could be done for example after each application or over a time series.

Although the samples have been described to be collected at different time points relative to a season, it should be understood that the samples may alternatively or additionally be collected when the relevant plant is in a particular growth stage. For example, the first or initial sample may be collected when the plant is at a tillering stage and a subsequent sample may be collected at a fruit development stage. More specifically, for cereal, the first or initial sample may be collected during BBCH 25-29 and the subsequent sample may be collected during BBCH 71-77.

Furthermore, the present disclosure is not limited to an initial sample and a single subsequent sample. Several subsequent samples may be collected at different time points and then processed so as to generate a resistance profile and/or virulence profile.

It is envisaged that the methods described herein can be used for any plant regardless of growing condition. For example, the methods can be applied to open field cultivated plants such as wheat, barley, potato, rice, fruit trees, grape, tomato, potato, cucurbits and many other cultivated crops by a farmer. The farmer, agriculturalist, agronomist or the like may pick a selection of specimens, such as leaves or any other part of the crop, soil or air surrounding the crop, across his field and process it as a single sample according to methods 100, 400 or 500, or send it to an organization for processing the sample according to methods 100, 400 or 500.

It is also envisaged that the methods described herein for determining at least one pesticide or other disease control measure (as described above) for a location can be applied to neighboring locations. This is based on the assumption that the variant(s) of the plant pathogen present in the location are also present in the neighboring locations. This may be the case for environmental aerial samples wherein the plants or crops are untreated with pesticides. It is well known that different situations can be found in neighboring locations, often due to different resistant plants or crops being cultivated and/or due to different treatment programs of pesticides, such as fungicides, having been adopted in previous years. In such cases, additional considerations need to be taken before applying disease control measures to a location that has been determined for an adjacent location.

In yet another example, the method 400 may alternatively or additionally further comprise determining, based on the DNA sequencing, the presence of sdhC_079N, sdhC_086S and/or sdhC_H152R genetic variants in plant pathogen Zymoseptoria tritici hosted by a wheat plant, and the method further comprises recommending a resistant wheat variety and/or a spray program comprising high intrinsic active SDHI or at least one fungicide with different mode of action including florylpicoaxamid, fenpicoxamid, metyltetraprole, mefentrifluconazole and/or prothioconazole as a disease control measure. Alternatively or additionally the method 400 may further comprise determining, based on the DNA sequencing, the presence of cyp51_379G, cyp51_381V, cyp51_460, cyp51_524T genetic variants in plant pathogen Zymoseptoria tritici hosted by a wheat plant, and the method further comprises recommending a resistant wheat variety and/or a spray program of high intrinsic active DMI or at least one fungicide with different mode of action including florylpicoxamid, fenpicoxamid, metyltetraprole, isoflucypram, fluxapyroxad, benzovindiflupyr or pydiflumetofen as a disease control measure.

One of the advantages of the methods described herein is the significant reduction in time for producing and analyzing the DNA sequencing data compared to other known methods where analyzing variants of plant pathogen typically takes weeks at which point the data is no longer a representation of the disease progression in real-time. The reduction in time is achieved by the use of third generation sequencers such as nanopore sequencing technology or single-molecule real-time sequencing as described above which can provide quantification of long DNA sequences of a sample or plant pathogen population within a few hours, such as 48 hours, such that a real-time, close to real-time understanding of the disease progression can be obtained. Additionally, by applying the methods described herein all variances as well as the combination of all variances can be monitored at the same time. The quantification of identified variances and genotypes provide a new set of accurate understanding of the plant pathogen population and its resistance and virulence profiles such that a more tailored treatment program for disease control and resistance management can be provided. The present disclosure offers a tool of reference to cope and implement the objective of initiative like the Farm to Fork of the European Green Deal aiming a reduction of 50% of the use of pesticide in the next years. Under such a scenario, the present disclosure will enable tailored profiling of pathogen populations informing farmers, agriculturalist, agronomist or the like about the best local recommendation. In case of multiple sampling the present disclosure offers the possibility to monitor the effectiveness of the decision taken, for example, the determined pesticide or other disease control measures, propose alternative disease control measures, highlight alerts concerning variants and resistance development or communicate suboptimal decisions.

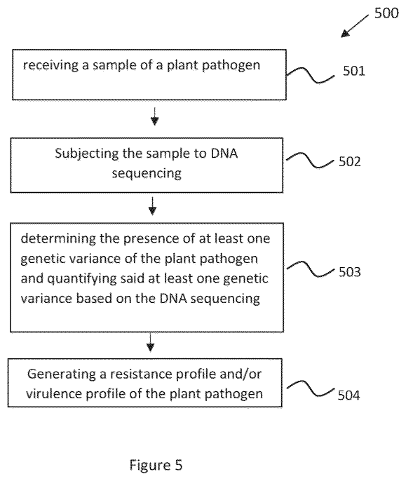

Another example of a method 500 according to the present disclosure will now be described with reference to FIG. 5. Any features described with reference to FIGS. 1 to 4, or any other examples or embodiments of the present disclosure, can be combined with method 500.

Method 500 is suitable for determining a resistance profile and/or virulence profile for a plant pathogen in a location according to the present disclosure. The method comprises receiving a sample of a plant pathogen from the location 501 wherein the samples could be a field sample or an environmental sample. In one example, operation 502 further comprises receiving the sample from a crop field. The method further comprises subjecting the sample to DNA sequencing 502 in order to determine the genetic sequence of bases of the plant pathogen. Based on the DNA sequencing, the presence of at least one genetic variance of the plant pathogen is determined and said at least one genetic variance is quantified 503. Thereafter, the method further comprises generating a resistance profile and/or virulence profile of the plant pathogen or pathogen population 504. This may be achieved by combining the results with information or knowledge obtained from dedicated studies assessing the impact of a given mutation on the performance of a specific fungicide or pesticide, or by evaluating the possible impact on crop productivity in case of increased virulence to the crop. Similar to method 100, method 500 enables the resistance profile and/or the virulence profile of a population of a pathogen present in a location to be determined rapidly such as in real-time, close to real-time, instantaneously or near instantaneously, within 48 hours or a few days. This provides opportunities to have a clear and more detailed understanding of the possible resistance profiles present in a location and to help determining a more tailored disease control measure and/or resistance management program for the current season and/or future seasons. As such, the most effective type of pesticide(s) for controlling the pathogen and or the best crop variety can be selected for current and future seasons such that pesticides with high presence of resistance can be avoided.

Optional features of method 500 will now be described.

The DNA sequencing of method 500 may provide a plurality of genetic variances, such as all genetic variance associated with resistance, and associated quantity of the plant pathogen in a single read, thereby providing rapid and comprehensive information on the disease.

Generating a resistance profile of method 500 may comprise associating the at least one genetic variance and corresponding quantity with a level of resistance to a pesticide and/or a group of pesticides. Generating a resistance profile may further comprise any of the features discussed in relation to FIGS. 1 to 4.

Generating a virulence profile of method 500 may comprise associating the at least one genetic variance and corresponding quantity with a level of virulence. The virulence profile or level indicates the plant pathogen's or a population of the plant pathogen's ability to invade a host and cause damage to different crop varieties. Virulence profiles indicating a high virulence to a specific crop might require a more intensive pesticide or fungicide application program. Generating a virulence profile may further comprise any of the features discussed in relation to FIGS. 1 to 4.

Method 500 may further comprise determining at least one disease control measure for controlling the development, reproduction and/or viability of the plant pathogen based on the resistance profile and/or virulence profile. The determined disease control measure may be at least one pesticide.

Method 500 may further comprise receiving a subsequent sample comprising the plant pathogen that has been subjected to at least one disease control measure, subjecting the sample to DNA sequencing, determining the presence of at least one genetic variance of the plant pathogen and quantifying said at least one genetic variance based on the DNA sequencing, generating a resistance profile and/or virulence profile of the plant pathogen, and comparing the resistance profile and/or virulence profile of the subsequent sample with that of the preceding sample. As described above, by comparing resistance profiles or virulence profiles based on two different time points, it is possible to determine if the pathogen population resistance to a fungicide class improved or deteriorated as a consequence of the agronomic decisions taken to control the disease.

In one example, based on the comparison, the method 500 may further comprise determining at least one pesticide or other disease control measure for controlling the plant pathogen. This pesticide or other disease control measure may be the same or different to the one(s) applied before the subsequent sample was received.

The initial sample may be collected at a tillering stage of a plant hosting the plant pathogen and the subsequent sample may be collected at a fruit development stage of said plant.

The sample and the subsequent sample may be obtained from a plurality of plants, spore traps and/or soil.

In one example of method 500, the sample may be obtained from the location, and the method further comprises determining at least one disease control measure for applying to a neighboring location based on the resistance and/or virulence profile.

Subjecting the sample to DNA sequencing as described in operation 602 may comprise using a sequencer capable of sequencing at least 200 base pairs, preferably at least 500 base pairs, in a single read.

The operation 602 may alternatively or additionally comprise using a sequencer configured to process at least 100 reads in a single experiment or run. Furthermore operation 602 may comprise using nanopore sequencing technology or single-molecule real-time sequencing.

In the event that method 500 determines the presence of sdhC_079N, sdhC_086S and/or sdhC_H152R genetic variants in plant pathogen Zymoseptoria tritici hosted by a cereal plant based on the DNA sequencing of operation 602, then the method 500 may further comprise recommending for example the adoption of resistant wheat varieties and/or inclusion in a spray program of high intrinsic active SDHI and/or other fungicides with different mode of action (e.g. florylpicoxamid, fenpicoxamid, metyltetraprole, mefentrifluconazole or prothioconazole) as a disease control measure

In the event that method 500 determines the presence of cyp51_379G, cyp51_381V, cyp51_460, cyp51_524T genetic variants in plant pathogen Zymoseptoria tritici hosted by a cereal plant based on the DNA sequencing of operation 602, then the method 500 may further comprise recommending for example the adoption of resistant wheat varieties and/or inclusion in a spray program of high intrinsic active DMI and/or other fungicides with different mode of action (e.g. florylpicoxamid, fenpicoxamid, metyltetraprole, isoflucypram, fluxapyroxad, benzovindiflupyr or pydiflumetofen) as a disease control measure.

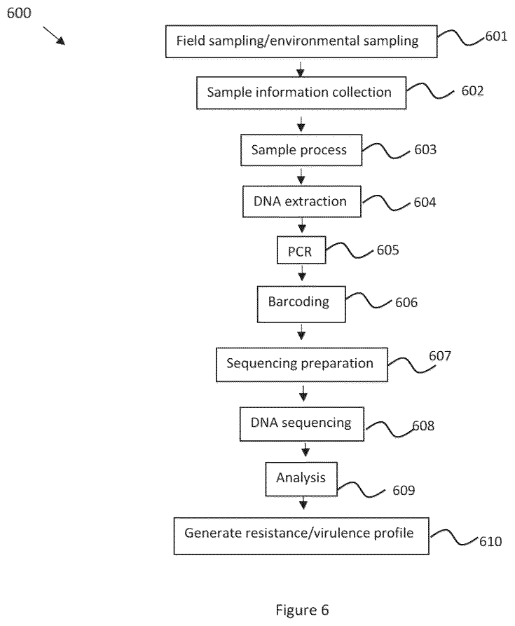

Examples of how methods 100, 400 and 500 can be implemented will now be described with reference to FIG. 6 illustrating a method 600 for determining a resistance and/or virulence profile for a plant pathogen in a location.

Method 600 comprises field sampling or environmental sampling 601 which involves collecting or picking specimens, such as leaves or any other part of the plant, soil or air surrounding the plant (e.g. through spore traps) from a location such as a field. If a resistance profile and/or virulence profile is to be determined for a plant pathogen, then specimens may be collected across the location at various points such that an average representation of the plant pathogen is obtained. The field sampling or environment sampling may correspond to the samples of methods 100, 400 and 500. Method 600 may further comprise sample information collection 602 which identifies where and when the sample was collected. Thereafter, the sample is received and sample processing 603 is performed on the sample which comprises bulking the collected specimens into a single sample such that it represents of a local population of the plant pathogen. Thereafter, DNA may be extracted 604 to create a DNA sample. Genes of interest are then amplified from the DNA sample by single step or multiplex PCR using specific primers 605. Thereafter, DNA barcoding 606 may be performed to identify the species of the plant pathogen using a short section of DNA from a specific gene or series of genes. The next operation may be sequencing preparation 607 followed by the operation of DNA sequencing 608 using a third-generation sequencer as described above. The DNA sequencing 608 determines the nucleic acid sequence and this data is then analyzed 609 to determine the presence of at least one genetic variance (or all) of the plant pathogen and quantifying each genetic variance based on the DNA quantification. Based on this analysis, a resistance profile and/or virulence profile of the plant pathogen is generated 610 by combining the results obtained from the DNA sequencing with knowledge or information on the different genetic variances and their ability of resistance to a specific pesticide or resistance trait of a crop.

Similar to methods 100, 400, 500 as described above, various parameters such as disease control measures can be determined based on the resistance and/or virulence profiles generated in operation 610 in order to control the plant pathogen in the current season and/or future seasons. For example, at least one pesticide can be determined that is expected to be the most effective in controlling the plant pathogen population whilst managing resistance. In addition, pesticides expected to have low performance in such situation can be avoided. Other parameters that can be considered during the determination of the pesticide or fungicide to be applied is the ability of the plant pathogen to overcome the resistance of the plant or crop in the field. Virulence profiles indicating a high virulence to a specific crop might require a more intensive pesticide or fungicide application program. Other parameters include time point of when to apply at least one pesticide, interval(s) between time points of applying at least one pesticide, nozzle size for applying pesticide and/or rate of applying pesticide.

As described in the present disclosure the resistance and virulence profiles generated according to any of the methods described herein have several applications including monitoring disease progression including resistance development of a plant pathogen, monitoring virulence of a plant pathogen relative to a specific plant variety, and providing tailored recommendations for disease control measures close to real-time or within a season. The recommendations for disease control measures are for controlling the development, reproduction and/or viability of a plant pathogen, in particular to manage resistance from developing. Furthermore, the resistance and/or virulence profiles of the present disclosure can also be used for monitoring adaptation of plant pathogens to biological agents.

The methods described herein are not limited to a particular plant or crop, growth condition, geographical region, plant pathogen or plant protection product such as a pesticide. The methods can be applied to any cultivated or wild crop where resistance development needs to be managed or monitored. For example, the methods can be applied to other patho-systems such as Pyrenophora teres and Ramularia collo-cygni in barley, Alternaria solani in potato and tomatoes, Pseudoperonospora cubensis in cucurbits, Plasmopara viticola in grapes, Peronospora destructor in onion, Plasmopara halstedii in sunflowers and many others in Europe and other geographies where mutations leading to reduced sensitivity to fungicides are known.

It should be noted that the above-mentioned examples illustrate rather than limit the invention, and that those skilled in the art will be able to design many alternative examples without departing from the scope of the appended claims. The word “comprising” does not exclude the presence of elements or steps other than those listed in a claim, “a” or “an” does not exclude a plurality. Any reference signs in the claims shall not be construed so as to limit their scope.

1. A method for determining a resistance and/or virulence profile for a plant pathogen in a location, the method comprising:
receiving a sample comprising a plant pathogen,
subjecting the sample to DNA sequencing,
determining the presence of at least one genetic variance of the plant pathogen and quantifying said at least one genetic variance based on the DNA sequencing; and
generating a resistance profile and/or virulence profile of the plant pathogen.

2. A method according to claim 1, wherein the DNA sequencing provides a plurality of genetic variances and associated quantity of the plant pathogen in a single read.

3. A method according to claim 1, wherein generating a resistance profile comprises associating the at least one genetic variance and corresponding quantity with a level of resistance to a pesticide and/or a group of pesticides.

4. A method according to claim 1, wherein generating a virulence profile comprises associating the at least one genetic variance and corresponding quantity with a level of virulence.

5. A method according to claim 1, further comprising determining at least one disease control measure for controlling the development, reproduction and/or viability of the plant pathogen based on the resistance profile and/or virulence profile.

6. A method according to claim 5, wherein the determined disease control measure is at least one pesticide.

7. A method according to claim 5, wherein the method further comprises, receiving a subsequent sample comprising the plant pathogen that has been subjected to at least one disease control measure, subjecting the sample to DNA sequencing, determining the presence of at least one genetic variance of the plant pathogen and quantifying said at least one genetic variance based on the DNA sequencing, generating a resistance profile and/or virulence profile of the plant pathogen, and comparing the resistance profile and/or virulence profile of the subsequent sample with that of the preceding sample.

8. A method according to claim 6, wherein the sample is collected at a tillering stage of a plant hosting the plant pathogen and the subsequent sample is collected at a fruit development stage of said plant.

9. A method according to claim 6, wherein the sample and the subsequent sample are obtained from a plurality of plants, spore traps and/or soil.

10. A method according to claim 1, wherein the sample is obtained from the location, and the method further comprises determining at least one disease control measure for applying to a neighboring location based on the resistance and/or virulence profile.

11. A method according to claim 1, wherein subjecting the sample to DNA sequencing comprises using a sequencer capable of sequencing at least 200 base pairs, preferably at least 500 base pairs, in a single read.

12. A method according to claim 1, wherein subjecting the sample to DNA sequencing comprises using a sequencer configured to process at least 100 reads in a single experiment.

13. A method according to claim 1, wherein subjecting the sample to DNA sequencing comprises using nanopore sequencing technology or single-molecule real-time sequencing.

14. A method according to claim 1, comprising determining, based on the DNA sequencing, the presence of sdhC_079N, sdhC_086S and/or sdhC_H152R genetic variants in plant pathogen Zymoseptoria tritici hosted by a wheat plant, wherein the method further comprises recommending a resistant wheat variety and/or a spray program comprising high intrinsic active SDHI or at least one fungicide with different mode of action including florylpicoaxamid, fenpicoxamid, metyltetraprole, mefentrifluconazole and/or prothioconazole as a disease control measure.

15. A method according to claim 1, comprising determining, based on the DNA sequencing, the presence of cyp51_379G, cyp51_381V, cyp51_460, cyp51_524T genetic variants in plant pathogen Zymoseptoria tritici hosted by a wheat plant, wherein the method further comprises recommending a resistant wheat variety and/or a spray program of high intrinsic active DMI or at least one fungicide with different mode of action including florylpicoxamid, fenpicoxamid, metyltetraprole, isoflucypram, fluxapyroxad, benzovindiflupyr or pydiflumetofen as a disease control measure.

16. A method according to claim 1, wherein receiving a sample comprising a plant pathogen comprises receiving from a crop field a crop sample comprising a plant pathogen.
